(19) United States
(12) Patent Application Publication
(10) Pub. No.: US 2026/0295210 A1
(43) Pub. Date: Oct. 1, 2026

(54) URINARY CATHETER ASSEMBLY WITH GUIDANCE DEVICE

(71) Applicant: Hollister Incorporated, Libertyville, IL (US)

(72) Inventors: David J. Farrell, Ballina (IE); David Pierce, Ballina (IE)

(21) Appl. No.: 19/477,376

(22) PCT Filed: Apr. 22, 2024
(86) PCT No.: PCT/US2024/025671

§ 371 Date: Oct. 21, 2025

Related U.S. Application Data
(60) Provisional application No. 63/497,923, filed on Apr. 24, 2023.

(51) Int. Cl. A61M 25/00

(52) U.S. Cl. CPC A61M 25/0017; A61M 25/0097; A61M 2205/586

(57) ABSTRACT

A urinary catheter product including a urinary catheter with a drainage member and a gripper. More particularly, a urinary catheter with a drainage member having a shorter axial length than the axial length of a gripper.

The present application claims the benefit of and priority to U.S. Provisional Application No. 63/497,923, filed Apr. 24, 2023, which is hereby incorporated herein by reference.

### FIELD OF THE INVENTION

This disclosure generally relates to a urinary catheter product including a gripper and a drainage member. More particularly, this disclosure relates to an intermittent urinary catheter product wherein the drainage member is shorter than the gripper.

### BACKGROUND

Intermittent catheterization is a good option for many users who suffer from various abnormalities of the urinary system. With the advent of intermittent urinary catheters, individuals with problems associated with the urinary system can conveniently self-catheterize to drain the individual's bladder. Individuals who suffer from urinary incontinence will self-catheterize several times a day.

Catheters may come in different lengths depending on the user. For instance, male catheters are typically 16 inches long. Female catheters may be shorter than male catheters. Generally, female catheters are 6-8 inches long.

Some catheter products may also include a funnel (or connector) and a gripper. A funnel/connector may extend from an end of a catheter tube and be used to direct urine to a waste receptacle. Grippers may be located on the catheter tube and are typically gripped by the user to assist in handling the catheter. For instance, a gripper may be used to assist in inserting the catheter. By using a gripper, a user may avoid having to handle the catheter tube.

Due to size and structural constraints, a gripper may not be suitable for use with some intermittent urinary catheters.

Therefore, there is a need for improved urinary catheter products that include a gripper.

### SUMMARY OF INVENTION

There are several aspects of the present subject matter which may be embodied separately or together in the subject matter claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a urinary catheter product is provided. The urinary catheter product includes a catheter tube having a catheter tube proximal insertion end and a catheter tube distal drainage end, wherein an inner surface defines a lumen. The urinary catheter product also includes a drainage member located at the catheter tube distal drainage end. The drainage member includes a drainage member proximal end and a drainage member distal end. The urinary catheter product also includes a gripper including an inner surface defining a lumen. The catheter tube is positioned within the lumen of the gripper. The gripper further includes a gripper proximal end and a gripper distal end, wherein the gripper distal end is abuttable with the drainage member proximal end and the drainage member has an axial length that is shorter than the axial length of the gripper.

### BRIEF DESCRIPTION OF DRAWINGS

FIG. 1A is a perspective view of an example of a catheter product with the gripper and drainage member in an abutted configuration.

FIG. 1B is a perspective view of the example of a catheter product of FIG. 1A with a gripper and a drainage member in a separated configuration.

FIG. 2A is a perspective view of an alternative catheter product with a gripper and a drainage member in an abutted configuration.

FIG. 2B is a perspective view of the alternative catheter product shown in FIG. 2A with a gripper and a drainage member in a separated configuration.

### DETAILED DESCRIPTION

A more detailed description of the device in accordance with the present disclosure is set forth below. It should be understood that the description of the specific devices below is intended to be exemplary, and not exhaustive of all possible variations or applications. Thus, the scope of the disclosure is not intended to be limiting and should be understood to encompass variations or embodiments that would occur to persons of ordinary skill.

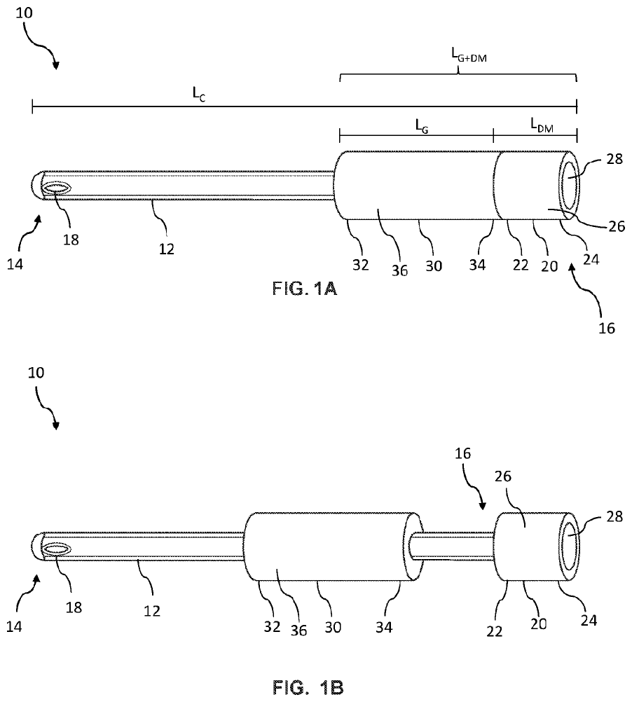

Turning to the Figures, FIGS. 1A and 1B show an example of a urinary catheter product 10, which may be an intermittent urinary catheter product. Urinary catheter product 10 includes a catheter tube 12 having a catheter tube proximal insertion end 14 and a catheter tube distal drainage end 16. It will be understood herein that proximal relates to a position closer to a user during use and distal relates to a position that is further from a user during use. A lumen extends from the catheter tube proximal insertion end 14 to the catheter tube distal drainage end 16. The catheter tube proximal insertion end 14 includes one or more openings 18 in communication with the lumen for receiving urine into the lumen. For instance, the one or more openings 18 for receiving urine may be an eyelet. In another example, the one or more openings 18 for receiving urine may be multiple eyelets. The one or more openings for receiving urine may vary in size, shape, and location without departing from the scope of the disclosure.

An outer surface of the catheter tube 12 may include a hydrophilic surface or coating. When wetted, the hydrophilic surface or coating may become lubricious, which may assist the user when inserting the catheter tube 12 into the urethra. In other alternatives, a lubricant, such as a gel lubricant, may be applied to the catheter tube 12 to assist inserting the catheter tube.

The catheter tube distal drainage end 16 includes a drainage opening for draining urine from the lumen. In an example, a drainage member 20 is located at the catheter tube distal drainage end 16. Drainage member 20 includes a drainage member proximal end 22 and a drainage member distal end 24. The drainage member 20 also includes an outer surface 26 and an inner surface 28 defining a lumen. The lumen extends from the drainage member proximal end 22 to the drainage member distal end 24. The lumen of the drainage member 20 is in fluid communication with the lumen of the catheter tube 12. The drainage member 20 may be, for example, but not limited to, a funnel or connector. The drainage member may be a separate component attached to the catheter tube drainage end 16. Alternatively, the drainage member 20 and catheter tube 12 may be integral. For example, the drainage member 20 and the catheter tube drainage end 16 may be a one-piece or unitary molded structure. When the drainage member 20 is a funnel, the drainage member 20 may have a similar circumference at both the drainage member proximal and distal ends 22, 24. In another example, the drainage member 20 may be tapered where the drainage member proximal end 22 has a smaller circumference than the drainage member distal end 24. In another instance, when the drainage member 20 is a connector, the connector may be connected to a drainage bag (not shown). Other drainage members known in the art may be used without departing from the scope of the disclosure.

Additionally, urinary catheter product 10 may include a gripper 30. A gripper 30 may be provided to assist the user with handling the catheter. For example, a user may grip the gripper 30 to handle and remove the catheter from the package and to aid in directing the insertion of the catheter tube 12 into the urethra.

Gripper 30 includes a gripper proximal end 32 and a gripper distal end 34. The gripper 30 also includes an outer surface 36 and an inner surface (not shown). The inner surface defines a lumen extending from the gripper proximal end 32 to the gripper distal end 34. The catheter tube 12 may be positioned within the lumen of the gripper 30. The outer surface 36 of the gripper 30 may be textured to aid in the user grip or as an indication of gripping location. The gripper 30 may be moveable along the length of the catheter tube 12. Furthermore, the gripper may be flexible or elastic such that the user may squeeze the gripper to grasp the catheter tube 12. When the user releases the gripper, the gripper substantially returns to its original shape to allow movement or sliding of the gripper along the catheter tube 12.

In a first configuration, shown in FIG. 1A, the gripper distal end 34 abuts the drainage member proximal end 22. In one alternative, the gripper distal end 34 and the drainage member proximal end 22 may be free ends that abut one another. In another alternative, the gripper 30 is releasably connected or connectable to the drainage member 20. For instance, the gripper distal end 34 may be connected to the drainage member proximal end 22 via a friction-fit, such as a push-fit connection. In another instance, the gripper 30 may be connected to the drainage member 20 via a snap-fit, a screw-fit, compression, an adhesive, or any other connection means known in the art. In yet another alternative, the gripper distal end 34 may be connected to the drainage member proximal end 22 by one or more frangible sections. For example, the gripper 30 and drainage member 20 may be a molded component that includes thin frangible sections between the gripper distal end 34 and the drainage member proximal end 22. Other releasable connections known in the art may be used without departing from the scope of the disclosure.

In one example, the gripper distal end 34 may correspond with the drainage member proximal end 22. For instance, the gripper distal end 34 may have a similar circumference or width as the drainage member proximal end 22. In another example, the drainage member proximal end 22 may be larger than the gripper distal end 34, and the drainage member proximal end 22 may be configured to receive the gripper distal end 34. In yet another example, the gripper distal end 34 may be larger than the drainage member proximal end 22, and the gripper distal end 34 may be configured to receive the drainage member proximal end 22.

Furthermore, the axial length L_{DM} of the drainage member 20 is shorter than the axial length L_{G} of the gripper 30. For example, the axial length L_{DM} of the drainage member 20 may be about 8-20 mm long and the axial length L_{G} of the gripper 30 may be about 12-44 mm or about 18-34 mm long. In some examples, the axial length L_{DM} of the drainage member 20 may be about 18%-50% of the axial length of the gripper. In other examples, the axial length L_{DM} of the drainage member 20 may be about 26%-37% of the axial length of the gripper. The combined axial length L_{G+DM} of the gripper 30 and drainage member 20 may be about 20-64 mm. For example, L_{G+DM} may be about 38 or 54 mm. In some examples, L_{G+DM} may be about 10%-40% of the axial length L_{C} of the catheter product 10. In other examples, L_{G+DM} may be about 25%-35% of the axial length L_{C} of the catheter product 10. The catheter tube may have a length of about 100-175 mm.

When the drainage member 20 and gripper 30 are in an abutted configuration, the abutted drainage member 20 and gripper 30 may provide a funnel having the appearance of a typical intermittent urinary catheter funnel. By having a drainage member 20 that is shorter than the gripper 30, the overall length of the catheter product 10 may be reduced while maintaining a sufficiently long catheter tube 12 for catheterization. This may make the catheter product 10 easier to handle. Additionally, by reducing the overall length of the catheter product 10 with a drainage member 20 that is shorter than the gripper 30, the amount of material needed to package the product may be reduced.

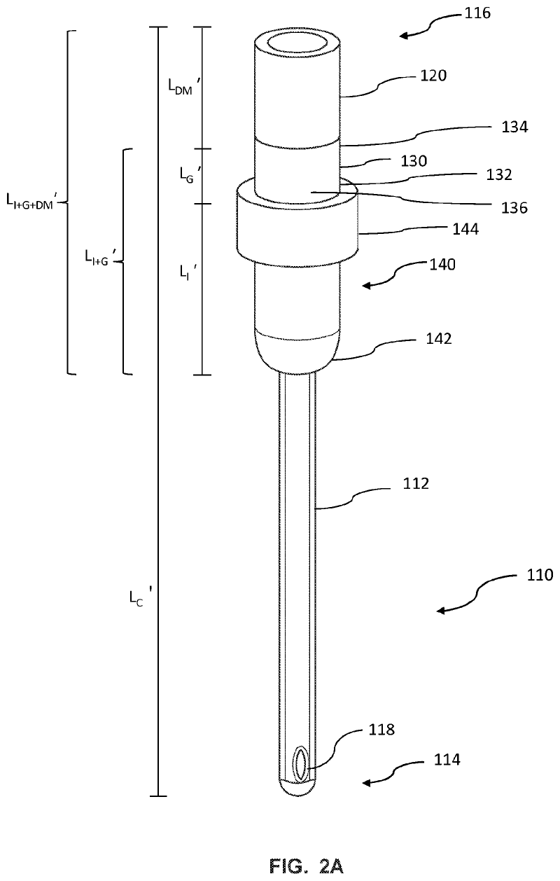

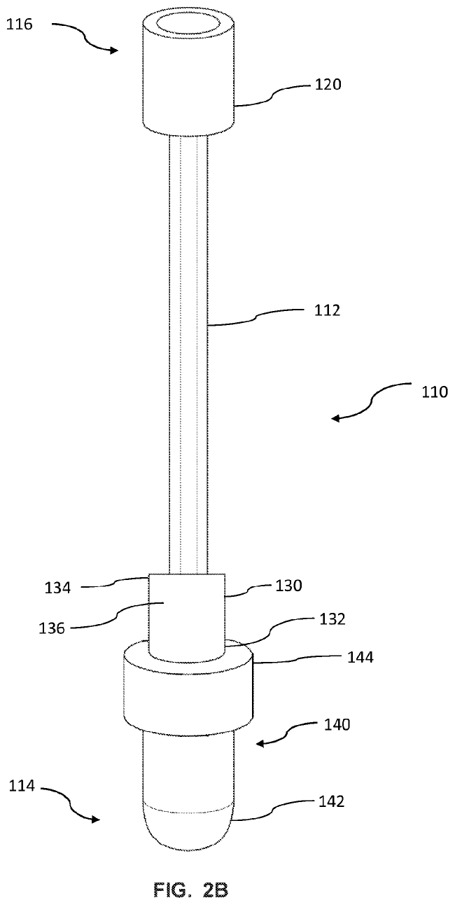

FIGS. 2A and 2B show another example of a catheter product 110 with a gripper 130 including an introducer tip 140. The catheter product 110 may have similar features and components as catheter product 10. Urinary catheter product 110 includes a catheter tube 112 having a catheter tube proximal insertion end 114 and a catheter tube distal drainage end 116. A lumen extends from the catheter tube proximal insertion end 114 to the catheter tube distal drainage end 116. The catheter tube proximal insertion end 114 includes one or more openings 118 for receiving urine in communication with the lumen.

Similar to urinary catheter product 10, urinary catheter product 110 includes a gripper 130 to assist the user with handling the catheter.

Gripper 130 includes a gripper proximal end 132 and a gripper distal end 134 and an outer surface 136 and an inner surface (not shown). The inner surface defines a lumen extending from the gripper proximal end 132 to the gripper distal end 134. The catheter tube 112 may be positioned within the lumen of the gripper 130. The gripper 130 may be moveable along the length of the catheter tube 112.

In this alternative, gripper 130 may include an introducer tip 140 located at the proximal end 132 of the gripper 130. The introducer tip 140 may assist the user in inserting the catheter tube 112 into the urethra. For instance, a user may grip a portion of the gripper 130 to move the introducer tip 140 towards the catheter tube proximal insertion end 114 to insert an introducer tip proximal end 142 into the urethra. The introducer tip 140 may include a cuff 144 to prevent the entire introducer tip 140 from entering the body. After the introducer tip 140 has been inserted, a user may then, while keeping the introducer tip 140 in place, grip the drainage member 120 to advance the catheter tube 112 through the introducer tip 140 and into the urethra. Other introducer tips known in the art may be used without departing from the scope of the disclosure.

Similar to gripper 30, the gripper 130 is abuttable to the drainage member 120. As shown in FIG. 2A, gripper 130 may abut drainage member 120 or may be connectable or connected to drainage member 120 in the manners described herein.

When an introducer tip 140 is included, the axial length L_{DM}′ of the drainage member 120 is shorter than the combined axial length L_{I+G}′ of the introducer tip 140 and the gripper 130. For example, the axial length L_{DM}′ of the drainage member may be about 6.75-20 mm long. Additionally, the axial length L_{G}′ of the gripper 130 may be about 12 mm and the axial length L_{I}′ of the introducer tip 140 may be about 15 mm. In some examples, the axial length L_{DM}′ of the drainage member 120 may be about 25%-43% of the combined axial length L_{I+G}′ of the introducer tip 140 and gripper 130. Furthermore, in some examples, the combined axial length L_{I+G+DM}′ of the introducer tip 140, gripper 130, and drainage member 120 may be about 16%-40% of the axial length L_{C}′ of the catheter product 110. In other examples, the combined axial length L_{I+G+DM}′ of the introducer tip 140, gripper 130, and drainage member 120 may be about 25%-35% of the axial length L_{C}′ of the catheter product 110. In an example, the combined axial length L_{I+G+DM}′ of the introducer tip 140, gripper 130, and drainage member 120 may be 30% of the axial length L_{C}′ of the catheter product 110. The catheter tube may have a length of about 100-175 mm.

When the drainage member 120 and gripper 130 are in an abutted configuration, the abutted drainage member 120 and gripper 130 provide a funnel that may have the size of a typical intermittent urinary catheter funnel. By having a drainage member that is shorter than the gripper, the overall length of the catheter product may be reduced while maintaining a sufficiently long catheter tube for catheterization. This may make the catheter easier to handle. Additionally, by reducing the overall length of the catheter product with a drainage member that is shorter than the gripper, the amount of material needed to package the product may be reduced.

It will be understood that the embodiments and examples described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

1. A urinary catheter product comprising:
a catheter tube having a catheter tube proximal insertion end and a catheter tube distal drainage end, wherein an inner surface of the catheter tube defines a lumen;
a drainage member located at the catheter tube distal drainage end, wherein the drainage member has a drainage member proximal end and a drainage member distal end; and
a gripper including an inner surface defining a lumen, wherein the catheter tube is positioned within the lumen of the gripper, the gripper having a gripper proximal end and a gripper distal end wherein the gripper distal end is abuttable to the drainage member proximal end and the drainage member having an axial length that is shorter than the axial length of the gripper.

2. The urinary catheter product of claim 1, wherein the gripper distal end is releasably connected to the drainage member proximal end.

3. The urinary catheter product of claim 2, wherein the gripper distal end is releasably connectable to the drainage member by a push-fit connection.

4. The urinary catheter product of claim 1, wherein the gripper is moveable along the length of the catheter tube.

5. The urinary catheter product of claim 4, wherein the gripper proximal end includes an introducer tip.

6. The urinary catheter product of claim 1, wherein the gripper includes a textured outer surface.

7. The urinary catheter product of claim 1, wherein the axial length of the drainage member is 18%-50% of the length of the gripper.

8. The urinary catheter product of claim 1, wherein the axial length of the drainage member is 26%-37% of the length of the gripper.

9. The urinary catheter product of claim 1, wherein the axial length of the drainage member is 6.75-20 mm.

10. The urinary catheter product of claim 1, wherein the axial length of the gripper is 12-44 mm long.

11. The urinary catheter product of claim 1, wherein the axial length of the gripper is 18-34 mm long.

12. The urinary catheter product of claim 1, wherein the axial length of the catheter tube is 100-175 mm.

13. The urinary catheter product of claim 1, wherein a combined axial length of the gripper and drainage member is 10%-40% of the catheter product.

14. The urinary catheter product of claim 1, wherein a combined axial length of the gripper and drainage member is 25%-35% of the catheter product.

15. The urinary catheter product of claim 1, wherein the drainage member proximal end corresponds to a width of the gripper distal end.

16. The urinary catheter product of claim 1, wherein the drainage member distal end is wider than the drainage member proximal end.

17. The urinary catheter product of claim 1, wherein the drainage member is a funnel.

18. The urinary catheter product of claim 1, wherein when the gripper and the drainage member are in a connected configuration the gripper and drainage member provide a funnel.

19. The urinary catheter product of claim 1, wherein the catheter tube includes at least one drainage opening located at the catheter tube proximal insertion end.

20. The urinary catheter product of claim 1, wherein the lumen is in fluid communication with the drainage member.
